(19) United States
(12) Patent Application Publication
(10) Pub. No.: US 2012/0046604 A1
(43) Pub. Date: Feb. 23, 2012

(54) TISSUE SUCTION DEVICE

(76) Inventors: Oz Cabiri, Macabim (IL); Asnat Raziel, Givatayim (IL)

(21) Appl. No.: 12/860,895

(22) Filed: Aug. 22, 2010

(51) Int. Cl. A61M 1/00

(52) U.S. Cl. 604/35

(57) ABSTRACT

A tissue suction device including a tissue retracting element including a pair of tissue contacting surfaces separated from each other by a gap, the tissue contacting surfaces having a round rim around their edges, and a suction conduit having an opening proximal to the tissue contacting surfaces and in fluid communication with the gap, the tissue retracting element having a mode of operation wherein suction applied through the opening sucks tissue adjacent the tissue retracting element so that the tissue is drawn into the gap around the rim towards the tissue contacting surfaces.

### FIELD OF THE INVENTION

The present invention generally relates to devices for tissue suction, and particularly to a device for retracting tissue for the purposes of attaching portions of tissue to one another, e.g., in a transoral gastroplasty procedure.

### BACKGROUND OF THE INVENTION

Gastroplasty procedures are known for treating obesity, gastroesophageal reflux disease (GERD), cancer, diabetes and the like. Gastric bypass procedures include the well-known Roux-En-Y procedure, as well as other techniques that reduce the size of the stomach and/or form restrictive barriers, pouches and the like in the stomach or other parts of the gastrointestinal tract.

Devices and methods have been developed for transoral gastroplasty. For example, the TOGA system of Satiety, Inc., Palo Alto, Calif., is a natural orifice procedure (through the mouth). An endoscope is inserted through the mouth into the stomach to allow the surgeon to view the procedure in the stomach. A retractor device is then introduced transorally into the stomach. The retractor device has pods with vacuum ports for acquiring tissue in the stomach by vacuum-sucking the tissue between the vacuum ports to form a length of pleated or folded tissue. A stapler is used to staple the tissue pleats together, thereby creating a pouch or otherwise reducing the volume of the stomach. The device and procedure are described in US Patent Application 20080091079 to Roth et al., the disclosure of which is incorporated herein by reference.

### SUMMARY OF THE INVENTION

The present invention seeks to provide an improved device for tissue suction, particularly for the purposes of attaching portions of tissue to one another, as is described more in detail hereinbelow. The device is particularly useful in a transoral gastroplasty procedure assembly for stapling or otherwise joining pleats of stomach tissue to each other, but the invention is not limited to transoral gastroplasty, and may be used in many laparoscopic, endoscopic, or natural orifice procedures in other organs of the body.

There is thus provided in accordance with an embodiment of the present invention a tissue suction device including a tissue retracting element including a pair of tissue contacting surfaces separated from each other by a gap, the tissue contacting surfaces having a round rim around their edges, and a suction conduit having an opening proximal to the tissue contacting surfaces and in fluid communication with the gap, the tissue retracting element having a mode of operation wherein suction applied through the opening sucks tissue adjacent the tissue retracting element so that the tissue is drawn into the gap around the rim towards the tissue contacting surfaces. The rim may be inwardly-directed, i.e., towards the gap.

In accordance with an embodiment of the present invention the pair of tissue contacting surfaces have an outer contour that forms part of an ellipsoid (e.g., a sphere) and an inner contour defined by a carved-out portion of the ellipsoid, the carved-out portion defining the gap.

In accordance with an embodiment of the present invention the tissue contacting surfaces are symmetric about the opening.

In accordance with an embodiment of the present invention the tissue contacting surfaces include two arcuate walls separated by the gap. The arcuate walls may include two ends of an oval surface extending upwards about a longitudinal axis that runs along the suction conduit.

There is also provided in accordance with an embodiment of the present invention a method including introducing the tissue retracting element distally into an organ of a body, and applying suction through the opening to suck tissue adjacent the tissue retracting element so that the tissue is drawn into the gap around the rim towards the tissue contacting surfaces. The tissue retracting element may be introduced through a natural orifice of the body, such as the mouth.

In accordance with an embodiment of the present invention the method further includes attaching portions of the tissue drawn into the gap to each other, such as by stapling the portions of the tissue to each other with a stapler introduced through a natural orifice of the body.

In accordance with an embodiment of the present invention the method further includes moving the tissue retracting element to different places in the organ as suction is applied and attaching portions of the tissue drawn into the gap to each other at the different places.

### BRIEF DESCRIPTION OF THE DRAWINGS

The present invention will be understood and appreciated more fully from the following detailed description taken in conjunction with the drawings in which:

FIGS. 1A-1D are simplified partially sectional, pictorial, front-view and bottom perspective illustrations, respectively, of a tissue suction device, constructed and operative in accordance with an embodiment of the present invention;

FIG. 2 is a simplified illustration of introducing the tissue suction device of FIGS. 1A-1D into a body organ (stomach);

FIG. 3 is a simplified head-on illustration of the tissue suction device in the body organ, before application of suction;

FIG. 4 is a simplified head-on illustration of the tissue suction device in the body organ, after application of suction;

FIG. 5 is a simplified illustration of attaching portions of tissue sucked into the tissue suction device (e.g., sutures or tacks), in accordance with an embodiment of the present invention;

FIG. 6 is a simplified illustration of the outer contour of the body organ after attaching portions of tissue sucked into the tissue suction device; and

FIG. 7 is a simplified illustration of using a stapler to staple portions of tissue sucked into the tissue suction device, in accordance with an embodiment of the present invention.

### DETAILED DESCRIPTION OF EMBODIMENTS

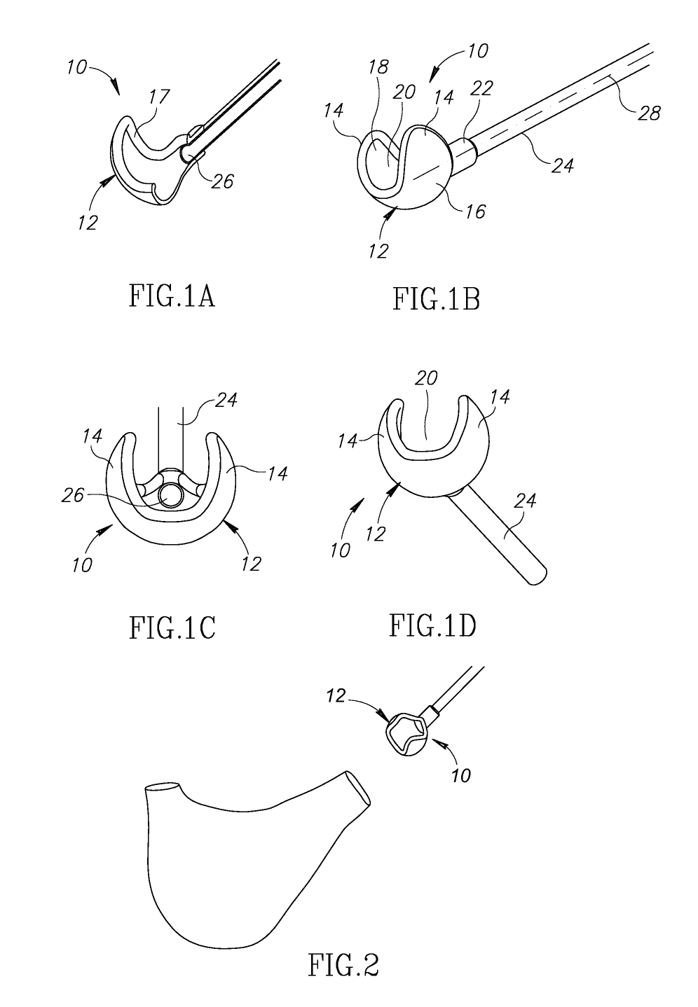

Reference is now made to FIGS. 1A-1D, which illustrate a tissue suction device 10, constructed and operative in accordance with an embodiment of the present invention.

Tissue suction device 10 includes a tissue retracting element 12 including a pair of tissue contacting surfaces 14 having an outer contour 16 that forms part of an ellipsoid and an inner contour 18 defined by a carved-out portion of the ellipsoid that defines a gap 20. The ellipsoid may be a sphere or curved object resembling a sphere. The curved surfaces of the ellipsoid are much better than other surfaces for drawing tissue in the gap for eventual attachment. A proximal neck 22 extending from tissue retracting element 12 is attached to a distal end of a suction conduit 24 having an opening 26 (FIGS. 1A and 1C). Opening 26 is proximal to the tissue contacting surfaces 14 and is in fluid communication with gap 20. The tissue contacting surfaces 14 may have an inwardly-directed round rim 17 around their edges (FIG. 1A). In contrast with the known prior art, the structure of inwardly-directed rim 17 (i.e., directed towards the gap) with its rounded edge helps in sealing the vacuum applied to the tissue being retracted into tissue retracting element 12, the tissue clinging around the round edged rim. An outwardly-directed round rim can also be used, or a rim that protrudes both inwards and outwards.

Tissue retracting element 12 may be constructed of any medically safe material, such as but not limited to, stainless steel, titanium alloy and different plastics, for example. As another example, tissue retracting element 12 may be constructed of an elastic material (or even shape memory material) and introduced in a contracted orientation into the patient and then expanded in-situ in the stomach, such as by inflating with liquid or gas introduced to element 12 or by expanding due to the shape memory properties. Another way of expanding would be to construct tissue retracting element 12 with joints; the element would be introduced in a contracted orientation into the patient and then expanded in-situ in the stomach by pulling with wires, in a manner similar to expanding a “ship in the bottle”.

In the non-limiting, illustrated embodiment, tissue contacting surfaces 14 include two arcuate walls symmetric about opening 26, separated by gap 20. The arcuate walls may include two ends of an oval surface extending upwards about a longitudinal axis 28 that runs along suction conduit 24.

Reference is now made to FIGS. 2-7 which illustrate using the tissue suction device 10 to retract and attach tissue, in accordance with an embodiment of the present invention. The method is shown and described for transoral gastroplasty, but the invention is not limited to the stomach or other parts of the GI tract, and may be used in other organs of the body.

In FIG. 2, tissue suction device 10 is introduced distally into the body organ (stomach), such as through the mouth of the body.

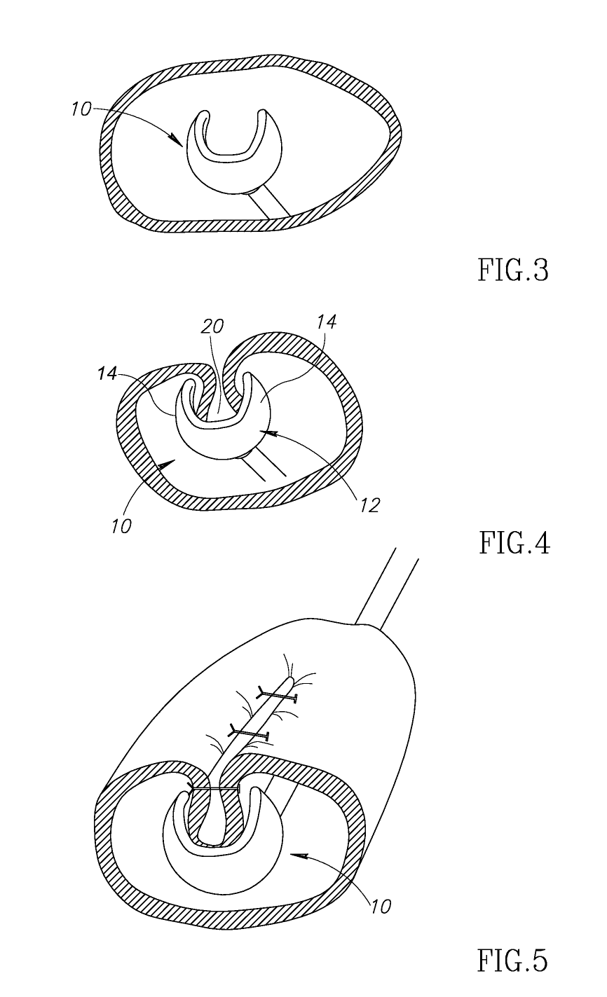

FIG. 3 illustrates tissue suction device 10 in the body organ, before application of suction.

FIG. 4 illustrates tissue suction device 10 after application of suction (the suction force works in the direction of the arrows). The suction is applied through opening 26 (not shown) to suck tissue adjacent tissue retracting element 12 so that the tissue is drawn into gap 20 towards the tissue contacting surfaces 14.

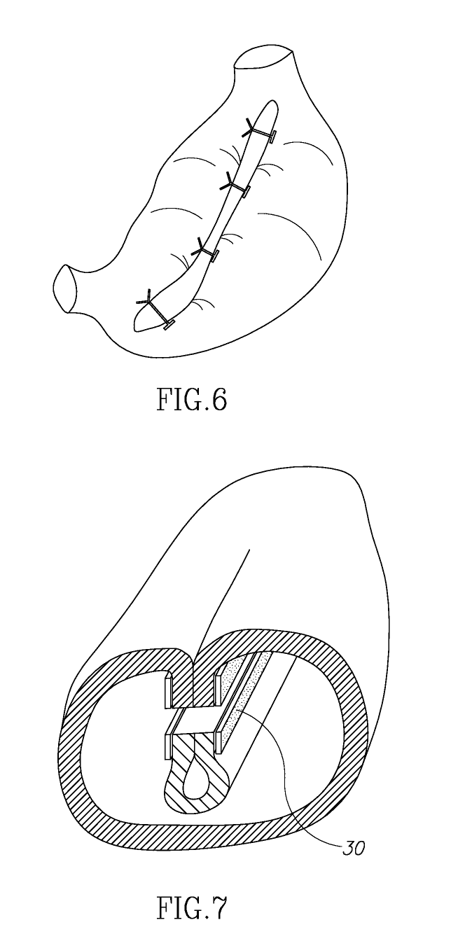

Reference is now made to FIG. 5. Portions of tissue sucked into tissue suction device 10 are attached to each other, such as with sutures or tacks, in accordance with an embodiment of the present invention. Many endoscopic tools for suturing or applying tacks (e.g., rotary tacks) are commercially available, such as from TYCO or STORZ. FIG. 6 illustrates the outer contour of the body organ after attaching the portions of tissue sucked into the tissue suction device.

In a preferred embodiment, as shown in FIG. 7, a stapler 30, introduced through a natural orifice of the body (e.g., the mouth), is used to staple portions of tissue sucked into the tissue suction device. An example of a commercially available stapler for use in this step is the ENDO GIA ULTRA UNIVERSAL endoscopic stapler from COVIDIEN.

In accordance with an embodiment of the present invention, tissue retracting element 12 is moved (e.g., proximally, distally or to the sides) to different places in the organ as suction is applied. This can be used to create a line along which the portions of the tissue drawn into the gap are attached to each other. The attached portions of tissue result in a reduced volume of the body organ, as desired.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A tissue suction device comprising:
a tissue retracting element comprising a pair of tissue contacting surfaces separated from each other by a gap, said tissue contacting surfaces having a round rim around their edges; and
a suction conduit having an opening proximal to said tissue contacting surfaces and in fluid communication with said gap, said tissue retracting element having a mode of operation wherein suction applied through said opening sucks tissue adjacent said tissue retracting element so that the tissue is drawn into said gap around said rim towards said tissue contacting surfaces.

2. The tissue suction device according to claim 1, wherein said pair of tissue contacting surfaces have an outer contour that forms part of an ellipsoid and an inner contour defined by a carved-out portion of the ellipsoid, the carved-out portion defining said gap.

3. The tissue suction device according to claim 1, wherein said tissue contacting surfaces are symmetric about said opening.

4. The tissue suction device according to claim 2, wherein said ellipsoid is a sphere.

5. The tissue suction device according to claim 1, wherein said tissue contacting surfaces comprise two arcuate walls separated by said gap.

6. The tissue suction device according to claim 5, wherein said arcuate walls comprise two ends of an oval surface extending upwards about a longitudinal axis that runs along said suction conduit.

7. The tissue suction device according to claim 1, wherein said rim is inwardly-directed towards said gap.

8. A method comprising:
introducing a tissue retracting element distally into an organ of a body, said tissue retracting element comprising a pair of tissue contacting surfaces separated from each other by a gap, said tissue contacting surfaces having a round rim around their edges, and a suction conduit having an opening proximal to said tissue contacting surfaces and in fluid communication with said gap; and
applying suction through said opening to suck tissue adjacent said tissue retracting element so that the tissue is drawn into said gap around said rim towards said tissue contacting surfaces.

9. The method according to claim 8, comprising introducing said tissue retracting element through a natural orifice of the body.

10. The method according to claim 9, wherein the natural orifice is a mouth of the body.

11. The method according to claim 8, further comprising attaching portions of the tissue drawn into said gap to each other.

12. The method according to claim 11, wherein attaching portions of the tissue to each other comprises stapling the portions of the tissue to each other.

13. The method according to claim 11, wherein attaching portions of the tissue to each other comprises stapling the portions of the tissue to each other with a stapler introduced through a natural orifice of the body.

14. The method according to claim 8, further comprising moving said tissue retracting element to different places in the organ as suction is applied and attaching portions of the tissue drawn into said gap to each other at the different places.
